(19) United States
(12) Patent Application Publication
(10) Pub. No.: US 2023/0365909 A1
(43) Pub. Date: Nov. 16, 2023

(54) BIOMASS MEASURING SYSTEM FOR FIXED BED BIOREACTOR AND RELATED METHODS

(71) Applicant: UNIVERCELLS TECHNOLOGIES S.A., Nivelles (BE)

(72) Inventors: José Castillo, Brussels (BE); Jean-Christophe Drugmand, Wavre (BE); Sebastien Rodriguez, Nivelles (BE); Julien Cardon, Nivelles (BE); Thomas Bouckenooghe, Nivelles (BE)

(21) Appl. No.: 18/021,987

(22) PCT Filed: Aug. 23, 2021
(86) PCT No.: PCT/EP2021/073262

§ 371 Date: Feb. 17, 2023

Related U.S. Application Data
(60) Provisional application No. 63/068,669, filed on Aug. 21, 2020.

(51) Int. Cl. C12M 1/12; C12M 1/34; C12M 1/00

(52) U.S. Cl. CPC C12M 25/18; C12M 41/36; C12M 41/40; C12M 41/06

(57) ABSTRACT

A bioreactor includes a fixed bed for culturing cells and a sensor system for sensing a density of the cells in the fixed bed. The sensor may be selected from the group comprising: (a) a sensor for measuring impedance across at least a portion of the fixed bed; (b) a flowmeter for detecting a rate of flow of liquid associated with the fixed bed; (c) a sensor for measuring a pressure differential in a flow of liquid through the fixed bed; (d) a monitor, such as a light sensor or microscope, for detecting light from a light source for projecting light on or in the fixed bed, wherein the detected or measured characteristic is indicative of cell density in the fixed bed; (e) a chemical sensor within the fixed bed for detecting a chemical indicative of cell density in the fixed bed. Related sensor arrangements, systems, and methods are also disclosed.

This application claims the benefit of U.S. Provisional Patent Application Ser. No. 63/068,669, filed Aug. 21, 2020, the disclosure of which is incorporated herein by reference.

### TECHNICAL FIELD

This document relates generally to the cell culturing arts and, more particularly, to a biomass measuring system for a fixed bed bioreactor and related methods.

### BACKGROUND

Certain cell culturing devices, such as bioreactors, use a fixed bed for the growth of suspension cells which become entrapped therein or for the growth of adherent cells which attach and grow thereon. These bioreactors suffer from the inherent inability of the user to estimate or measure cell biomass within the fixed bed. For accurate cell density analysis, users often take samples of the fixed bed in which cells have grown during the culture process for analytical purposes (e.g., to take cell-associated measurements such as those relating to viability and density). Currently, the only way to take such an in-process sample from the fixed bed is to remove a portion or sample of the bed such as by reaching inside the bioreactor with a tool, such as a tweezer, to manually extract a piece or portion of the bed. This operation requires careful dexterity and invariably causes undesirable fluidic perturbations that risk disrupting the cell culture environment.

Furthermore, as the bioreactor will be in an open-phase during such extraction procedure, and thus to maintain the necessary sterile conditions, the bioreactor would typically be located inside a containment unit such as a laminar flow cabinet or a biosafety cabinet where the freedom of movement of the operator is limited. While a small-scale bioreactor can be placed in such a containment unit, a large production-scale version cannot readily be placed in such cabinets to achieve this result. Additionally, sterility must be maintained during the whole operation, which means the operator collecting the sample has to follow precise aseptic operating procedures. This is challenging when having to introduce an extraction tool.

Biomass sensors have been proposed for assessing cell density within a fixed bed bioreactor. However, these sensors lack sufficiently robust technology, and do not allow for the actual direct examination of the cells. Also, disposable (single use) bioreactors are used more readily in labs and manufacturing facilities today. Such single use bioreactors are manufactured of plastics which are flexible compared to glass or stainless steel used in traditional bioreactors. This flexible nature permits movement of the plastic walls and/or lid of such single use bioreactors, such as for example when pressure changes occur within the bioreactor. Such biomass sensors may prove inaccurate as they may move with the walls and/or lid during sensing, displacing their position within the fixed bed. Thus, current samplers, sensors and methods do not provide an accurate and timely tool for developing a reliable cell culture process for a fixed bed bioreactor.

Accordingly, a need is identified for a manner in which to assess the cell growth, such as by measuring cell density in the fixed bed in a reliable, repeatable/reproducible and accurate manner, while maintaining aseptic conditions so as to protect against contamination (both internal to the bioreactor and external to it) and to avoid creating deleterious disruptions of the fixed bed cell culture environment.

### SUMMARY

According to a first aspect of the disclosure, an apparatus for culturing cells is provided. The apparatus comprises a bioreactor including a fixed bed for culturing cells. The apparatus further includes a sensor system for sensing a cell density of at least a portion of the fixed bed. The sensor system may be one or more of the following: (a) a sensor for measuring impedance across at least a portion of the fixed bed; (b) a flowmeter for detecting a rate of flow of liquid associated with the fixed bed; (c) a sensor for measuring a pressure differential in a flow of liquid through the fixed bed; (d) a monitor, such as a light sensor or microscope, for detecting light from a light source for projecting light on or in the fixed bed; or (e) a chemical sensor for detecting a chemical indicative of the cell density in the fixed bed.

In one embodiment, the sensor system comprises the sensor for measuring impedance across at least a portion of the fixed bed. The impedance sensor comprises a pair of electrodes arranged with the portion of the fixed bed positioned therebetween.

In one embodiment, the sensor system comprises the flowmeter for detecting a rate of flow of liquid associated with the fixed bed. The flowmeter may be located within the fixed bed.

In one embodiment, the sensor system comprises the sensor for measuring a pressure differential in a flow of liquid through the fixed bed. The pressure differential sensor may comprise a first pressure sensor adjacent an entrance to the fixed bed and a second pressure sensor adjacent to an exit of the fixed bed.

In one embodiment, the sensor system comprises the monitor, such as the light sensor or microscope, for detecting light from a light source for projecting light on or in the fixed bed. The light source may comprise an optical fiber extending within the fixed bed.

In one embodiment, the sensor system comprises the chemical sensor within the fixed bed for detecting a chemical indicative of the cell density in the fixed bed.

According to another aspect of the disclosure, an apparatus for culturing cells comprises a bioreactor including a fixed bed for culturing cells and a sensor for measuring impedance across at least a portion of the fixed bed. The sensor may comprise a pair of electrodes having the portion of the fixed bed positioned therebetween.

According to another aspect of the disclosure, an apparatus for culturing cells comprises a bioreactor including a fixed bed for culturing cells and a flowmeter for detecting a flow rate of liquid through the fixed bed. The flowmeter may be located within the fixed bed.

According to another aspect of the disclosure, an apparatus for culturing cells is provided. The apparatus comprises a bioreactor including a fixed bed for culturing cells and a sensor for sensing a pressure differential in a flow of liquid through the fixed bed. The sensor may comprise a first pressure sensor located adjacent to an entrance of the fixed bed and a second pressure sensor located adjacent to an exit of the fixed bed.

According to another aspect of the disclosure, an apparatus for culturing cells, comprises a bioreactor including a fixed bed for culturing cells and a monitor for monitoring light from a light source for projecting light on or in the fixed bed. The light source comprises an LED for projecting light within the fixed bed, and/or an optical fiber located within the fixed bed. The monitor may comprise a microscope or a light sensor. In one particular version, the light source comprises a UV lamp, and the monitor comprises a fluorescence detector.

According to another aspect of the disclosure, an apparatus for culturing cells comprises a bioreactor including a fixed bed for culturing cells and a chemical sensor within the fixed bed for detecting a chemical indicative of cell density in the fixed bed.

In any of the foregoing embodiments, the fixed bed may comprise a cell growth matrix assembly having one or more cell immobilization layers having a surface which allows cells to adhere and grow, and one or more spacer layers containing a tortuous path producing structure adjacent to said cell immobilization layers, allowing passage of cells and medium along the surface of both the one or more cell immobilization and the one or more spacer layers but in a tortuous path wherein the cells will efficiently travel into the one or more cell immobilization layers and adhere at a depth therein. The bioreactor may comprise an annular housing including a chamber for receiving the fixed bed. The fixed bed may comprise a plurality of woven layers. The fixed bed may comprises a plurality of woven layers in a vertical stack, and arranged such that a flow of liquid is in a transverse direction. The fixed bed may comprise a monolithic matrix, such as one formed by 3-D printing.

According to another aspect of the disclosure, an apparatus for culturing cells a bioreactor including a fixed bed for culturing cells and a biomass sensor associated with a portion of the fixed bed. The portion may be located in a common chamber with the fixed bed. The portion may comprise a representative portion of the fixed bed and is located in a chamber of the bioreactor different from the chamber including the fixed bed. The biomass sensor comprises a probe supported by a lid of the bioreactor.

The portion of the portion of the fixed bed may comprise a discrete piece located external to the bioreactor. Specifically, the discrete piece may be located in a chamber external to the bioreactor associated with a circulation loop for transmitting liquid from the bioreactor to the chamber and returning the liquid from the chamber to the bioreactor.

According to another aspect of the disclosure, a biomass sensor includes a receiver for receiving a cell culture material. In one embodiment, the receiver comprises a basket. A bioreactor may include a fixed bed in one chamber and the biomass sensor in another chamber.

According to another aspect of the disclosure, a bioreactor includes a fixed bed including a cell culture material and having a liquid permeable receiver including a portion of the cell culture material of the fixed bed. The liquid permeable receiver may be located in an opening in the fixed bed formed by the removal of the portion of the cell culture material. The liquid permeable receiver may be removable. The portion of the cell culture material in the liquid permeable receiver includes an opening for receiving a biomass sensor.

According to another aspect of the disclosure, a method for sensing biomass associated with a bioreactor including a fixed bed for culturing cells is provided. The method comprises one or more of the following steps: (a) measuring impedance across at least a portion of the fixed bed; (b) detecting a rate of flow of liquid associated with the fixed bed; (c) measuring a pressure differential in a flow of liquid through the fixed bed; (d) detecting light from a light source for projecting light on or in the fixed bed; or (e) detecting a chemical indicative of the cell density in the fixed bed.

In one embodiment, the step comprises measuring impedance across at least the portion of the fixed bed.

In one embodiment, the step comprises detecting the rate of flow of liquid associated with the fixed bed.

In one embodiment, the step comprises measuring the pressure differential in the flow of liquid through the fixed bed.

In one embodiment, the step comprises detecting light from a light source for projecting light on or in the fixed bed.

In one embodiment, the step comprises detecting a chemical indicative of the cell density in the fixed bed.

According to a further aspect of the disclosure, a method for sensing biomass associated with a bioreactor including a fixed bed for culturing cells. The method comprises providing a biomass sensor at least partially within the bioreactor carrying a portion of the fixed bed. The method may comprise the step of removing the biomass sensor and the portion from the bioreactor.

### BRIEF DESCRIPTION OF THE DRAWING FIGURES

FIG. 1 is a perspective view of an exemplary bioreactor for which certain aspects of this disclosure may have applicability;

FIG. 2 is a partially exploded view of further details of the bioreactor of FIG. 1;

FIG. 3 illustrates a structured fixed bed in spiral form for possible use in connection with a bioreactor;

FIGS. 3A, 3B, and 3C illustrate particular details of one example of a spiral fixed bed;

FIGS. 3D, 3E and 3F illustrate alternative arrangements for forming a structured fixed bed;

FIG. 3G illustrates another form of structured fixed bed;

FIGS. 4 and 5 schematically illustrates one possible embodiment of a biomass sensor system;

FIG. 6 illustrates the biomass sensor system of FIGS. 4 and 5 applied to a bioreactor including one or more fixed beds;

FIG. 7 illustrates a further embodiment of a of a biomass sensor system for a bioreactor including a fixed bed;

FIG. 8 illustrates a yet a further embodiment of a of a biomass sensor system for a bioreactor including a fixed bed;

FIG. 9 illustrates still a further embodiment of a of a biomass sensor system for a bioreactor including a fixed bed;

FIGS. 10, 11, and 12 illustrate another embodiment of a biomass sensor system for a bioreactor including a fixed bed;

FIG. 13 illustrates a biomass sensor system associated with an external loop connected to a bioreactor including a fixed bed;

FIGS. 14 and 15 illustrate another embodiment of a biomass sensor system for a fixed bed bioreactor;

FIG. 16 illustrates yet another arrangement of a sensor system for sensing biomass in a fixed bed bioreactor; and

FIGS. 17 and 18 illustrate further embodiments of arrangements for sensing biomass in association with a fixed bed of a bioreactor.

### DETAILED DESCRIPTION

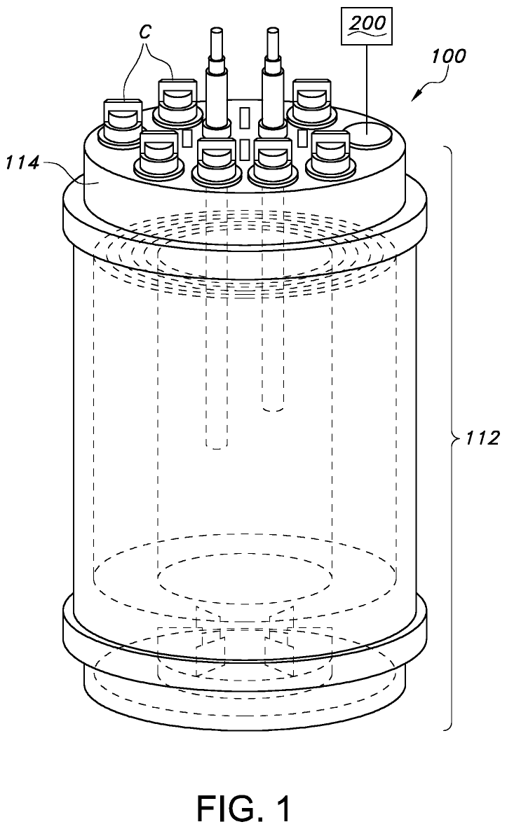

Reference is now made to FIGS. 1-3, which illustrate one embodiment of a fixed bed bioreactor 100 for culturing cells associated with a biomass measuring system 200, according to one aspect of the disclosure. In some embodiments, the bioreactor 100 includes an external casing or housing 112 forming or including an interior compartment and a cover 114 placed on top of the housing 112 to cover or seal the interior compartment after it is populated with at least the fixed bed. In an embodiment, the cover 114 is removable. The cover 114 may include various openings or ports with removable closures or caps C for allowing for the selective introduction or removal of material, fluid, gas, probes, sensors, samplers, or the like.

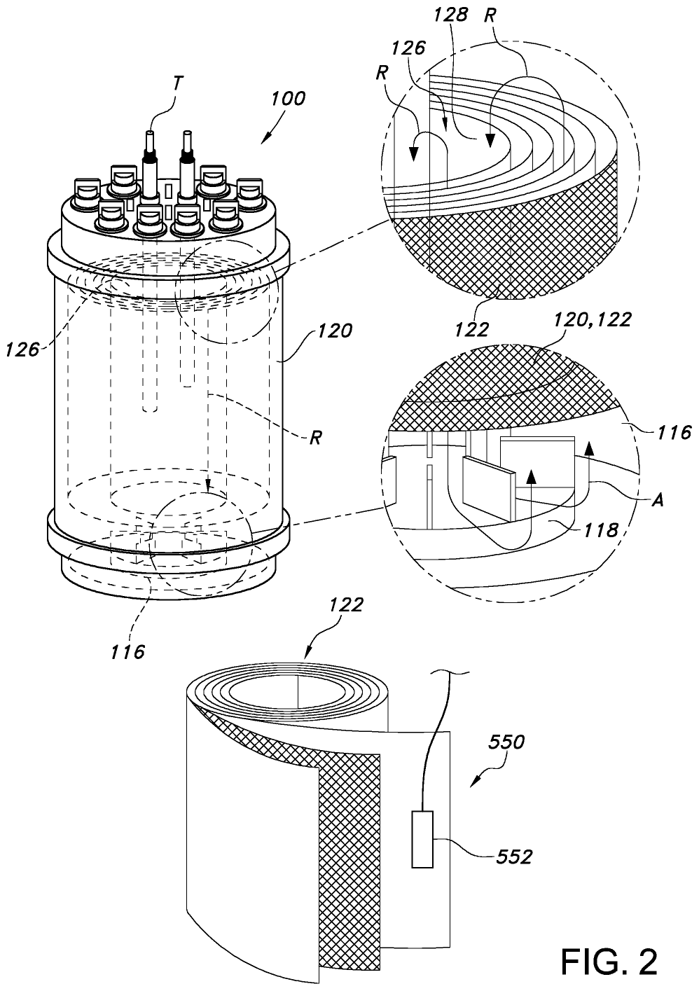

Within the interior compartment of the bioreactor housing 112, several compartments or chambers may be provided for transmitting a flow of fluid, gas, or both, throughout the bioreactor 100. As indicated in FIG. 2, in some embodiments, the chambers may include a first chamber 116 at or near a base of the bioreactor 100. In some embodiments, the first chamber 116 may include an agitator for causing fluid flow within the bioreactor 100. In some embodiments, the agitator may be in the form of a drop-in, rotatable, non-contact magnetic impeller 118, which thus forms a centrifugal pump in the bioreactor. The agitator could also be in the form of an impeller with a mechanical coupling to the base, an external pump forming part of a fluid circulation system, or any other device for causing fluid circulation within the bioreactor.

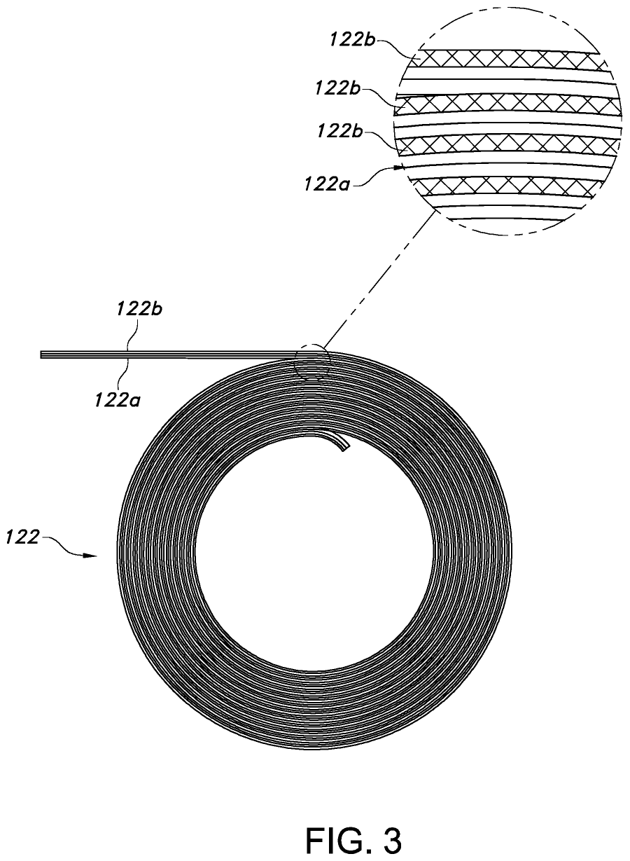

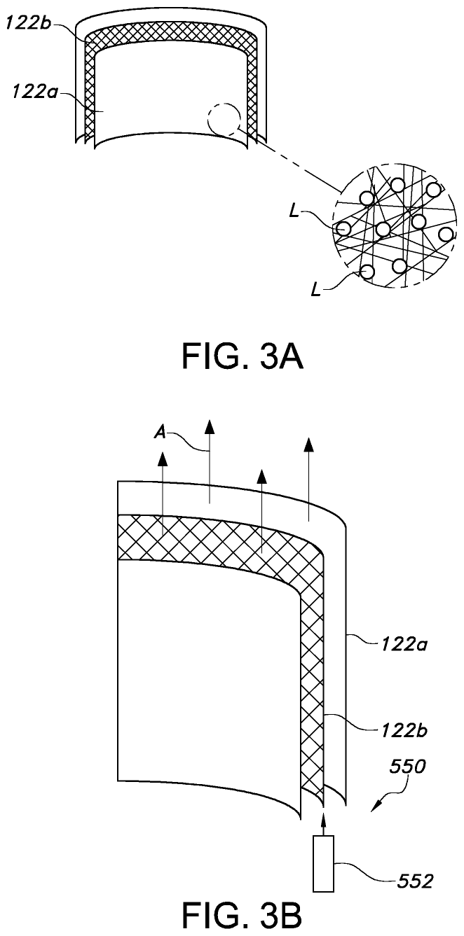

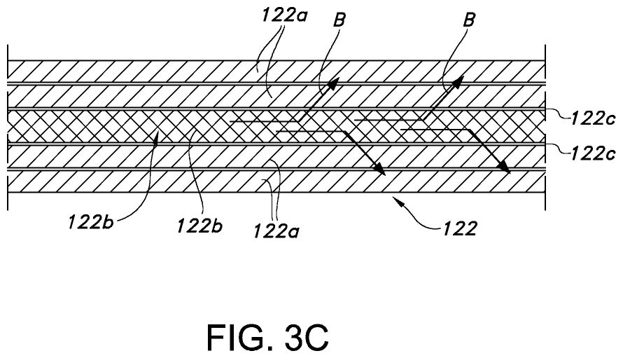

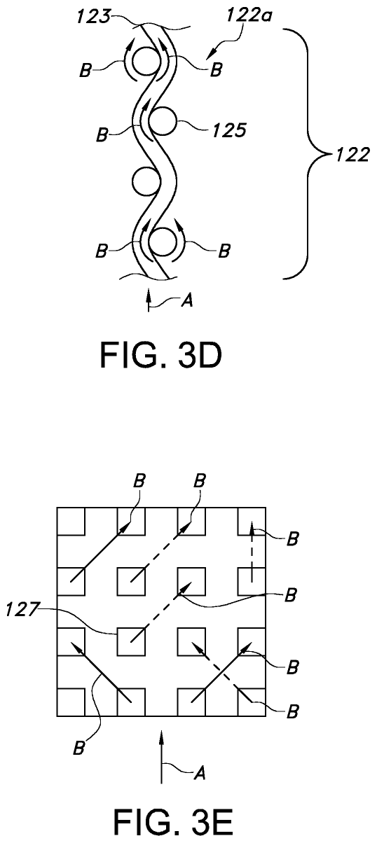

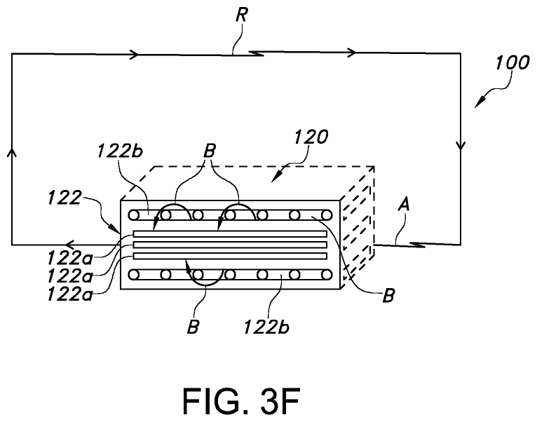

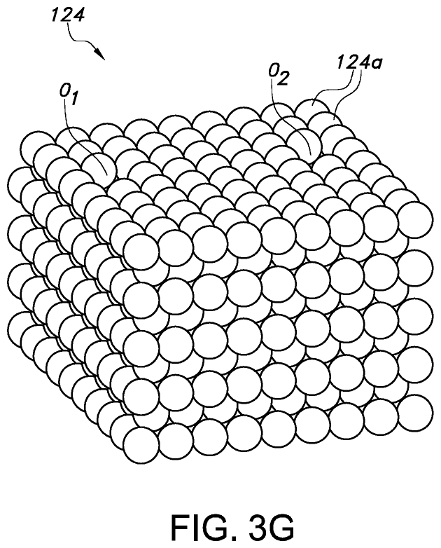

In some embodiments, as a result of the agitation provided, fluid may then flow upwardly (as indicated by arrows A in FIG. 2) into a chamber 120 along the outer or peripheral portion of the bioreactor 100 (or otherwise through the fixed bed). FIG. 3 shows a fixed bed in the form of a structured spiral bed 122 which, in use, may contain and retain cells being grown. In some embodiments, the spiral bed 122 may be in the form of a cartridge that may be built within and as a part of or introduced into the outer chamber 120. The bed 122 can be pre-installed in the chamber during manufacture at a facility prior to shipping or installed at the point of use. Other forms of fixed beds, such as structured or packed beds, may be used instead of the version shown in the drawings.

In some embodiments, fluid exiting the chamber 120 is passed to a headspace formed in a zone between one (upper) side of the bed 122 and the cover 114, where the fluid (media) is exposed to a gas (such as oxygen). In some embodiments, fluid may then flow radially inwardly to a central chamber 126 to return to the lower portion of bed 122. In some embodiments, this central chamber 126 can be columnar in nature and may be formed by an imperforate conduit or tube 128 or rather formed by the central opening of the structured spiral bed.

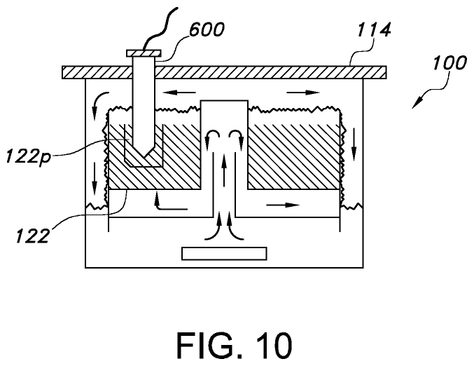

In some embodiments, the chamber 126 returns the fluid to the first chamber 116 (return arrow R) for recirculation through the bioreactor 100, such that a continuous loop results (bottom to top in this version). In some embodiments, a sensor, for example a temperature probe or sensor may also be provided for sensing the temperature of the fluid flowing or residing in the chamber 126. In some embodiments, additional sensors (such as, for example, pH, oxygen, dissolved oxygen, temperature, cell density, etc.) may also be provided at a location before the fluid enters (or re-enters) the chamber 116. However, as can be understood by the skilled artisan, this is merely an exemplary arrangement, and other forms of bioreactor exist (including, for instance, the version shown in FIGS. 10-13).

FIG. 3 shows one embodiment of a matrix material for use as a structured fixed bed in the bioreactor of the present disclosure and, in particular, one in the form of a spiral bed 122. In some embodiments, the spiral bed may comprise one or more cell immobilization layers 122a or structures. In another embodiment, the cell immobilization layers or structures may comprise one or more woven or non-woven layers or a combination of the foregoing.

In one embodiment, the cell immobilization layers 122a may be provided adjacent to one or more spacer layers 122b or structures. The spacer layers 122b made from a mesh or woven structure. In some embodiments, the layering may optionally be repeated several times to achieve a stacked or layered configuration.

In some embodiments, the mesh structure included in spacer layers 122b forms tortuous paths to steer the cells into the depth of the cell immobilization layers 122a (see cells L in FIG. 3A suspended or entrapped in the material of the immobilization layer 122a). As shown in FIGS. 3B and 3C, the spacer layers 122b also form channels 122c in conjunction with the adjacent cell immobilization layers 122a for fluid and bubbles to flow therethrough (see arrows A in FIG. 3B indicative of flow between layers, and also arrows B in FIG. 3C indicative of transverse flow). Increased homogeneity of the cells is maintained within the structured fixed bed as a result of this type of arrangement. In some embodiments, other spacer structures can be used which form such tortuous paths.

In some embodiments, as shown in FIGS. 3, 3A and 3B, the structured fixed bed can be subsequently spirally or concentrically rolled along an axis or core (e.g., conduit or tube 128, which may be provided in multiple component parts). In some embodiments, the layers of the structured fixed bed are firmly wound. In some embodiments, the diameter of the core, the length and/or amount of the layers will ultimately define the size of the assembly or matrix. In some embodiments, thickness of each of the layers 122a, 122b may be between 0.1 and 5 mm, 0.1 and 10 mm, or 0.001 and 15 mm.

In some embodiments, other structures can be used which form such tortuous paths. For example, FIG. 3D shows that the one or more cell immobilization layers 122a may be adapted to form a structured fixed bed 122. The one or more layers 122a provide a tortuous channel of flow (arrow B) from a linear or regular inflow (arrow A) without using additional spacer layers (but such may be used, if desired). This may be achieved, for example, by providing one or more layers of woven fibers or filaments 123, 125 that disrupt the flow.

FIG. 3E shows that such a result may be achieved using a non-woven material as the cell immobilization layer 122a. This may be achieved by forming the layer 122a as a reticulated arrangement (such as by 3-D printing) with openings 127 through which liquid may pass and return again, thus forming the tortuous channels that again promote homogeneity and also serve to further shear or divide any bubbles present in the liquid. This function may again be achieved with or without added spacer layers being present.

The orientation of the structured fixed bed 122 may be other than as shown in a bioreactor 100 as shown in FIG. 2, where the flow is arranged vertically (bottom to top, in the example provided). For example, as shown in FIG. 3F, a bioreactor 100 may include a first chamber 120 that includes a structured fixed bed 122 comprised of one or more horizontally arranged material layers. The one or more layers may comprise a woven or reticulated material, as per FIGS. 3D and 3E, but as illustrated in FIG. 3F, may comprise one or more cell immobilization layers 122a (three shown, but any number may be present) sandwiched by adjacent spacer layers 122b (vertical spacing exaggerated for purposes of illustration), which are optional.

The flow is thus arranged from side-to-side (left to right or right to left), with the material layer(s) (spacer or otherwise) providing for the channels for creating the tortuous flow (arrows B) from a linear or regular inflow (arrow A). The pumping action may be provided by an agitator or other pump at the entrance end of the chamber 120, and a return path provided at the exit end, as schematically illustrated by path R. Additional spacer layers may also be provided between the cell immobilization layers 122a, if desired.

In another possible embodiment, and with reference to FIG. 3G, the structured fixed bed 122 comprises a three-dimensional (3D) monolith matrix 124. The matrix 124 may take the form of a scaffold or lattice formed of multiple interconnected units or objects 124a (e.g., round or spherical beads connected by connectors), which objects have surfaces for cell adhesion. The matrix 124 may include a tortuous path for fluid and cells to flow therethrough when in use. In some embodiments, the matrix 124 may be in the form of a 3D array, lattice, scaffolding, or sponge. The matrix 124 may be single use in nature to avoid the cost and complexities involved in cleaning according to bioprocessing standards.

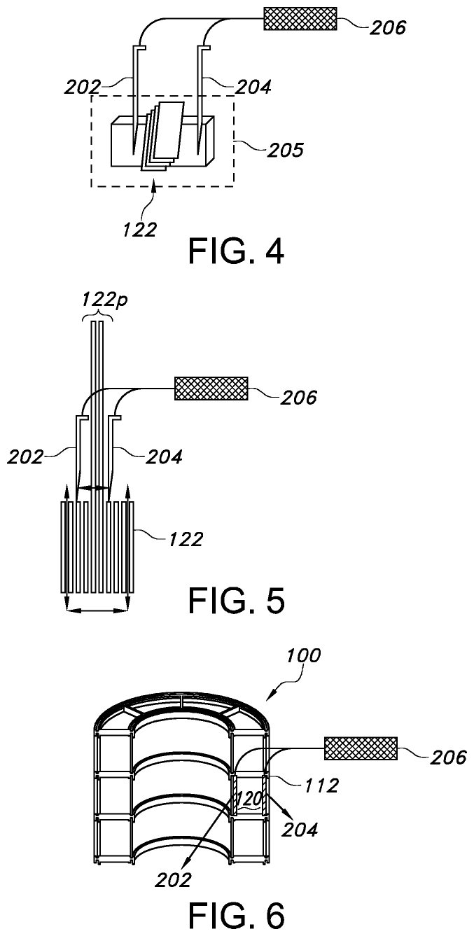

According to a first aspect of the disclosure, the biomass measuring system 200 may use an electrical signal, such as impedance, capacitance, or other dielectric signal, to assess cell density in order to measure biomass. In one embodiment, with reference to FIGS. 4-6, this may be achieved by providing two isolated electrodes 202, 204 connected to any device for measuring impedance, conductance, or capacitance, such as for example a volt-ohm meter 206. In the illustrated embodiment, the electrodes 202, 204 are shown as being probes in the form of elongated rods or pins, but may take other forms, such as plates, rings, or the like.

The electrodes 202, 204 are located at different locations within the cell culture area of the container 205, e.g., on different sides of the fixed bed 122 (which may be inside of the container 205, which may comprise a bioreactor) or possibly within the fixed bed 122 (such as between layers 122a, 122b, or in openings O1, O2 formed in the monolith matrix 124 shown in FIG. 3F).

In the example of FIG. 4, such fixed bed 122 takes the form of a plurality of layers, and electrodes 202, 204 are located on opposite sides of the fixed bed. The positioning may be such that the electrodes 202, 204 are closely spaced to the outermost sides of the fixed bed 122, or the electrodes 202, 204 may be spaced from the sides, as shown. A skilled artisan can appreciate that the positioning of the electrodes 202, 204 may be adjusted depending on the size, shape, or composition of the fixed bed 122.

In FIG. 5, electrodes 202, 204 are shown to be associated with an auxiliary portion 122p of the fixed bed 122 having cell growth conditions representative of the remainder of the fixed bed. As can be appreciated, this avoids the need for disrupting the fixed bed itself and thus promotes homogeneity.

In FIG. 6, electrodes 202, 204 are shown to be located on opposite sides (e.g., inner and outer) in a chamber of a bioreactor 100 including the fixed bed 122. In this embodiment, electrodes 202, 204 should be electrically isolated from the adjacent walls of the housing 112 to ensure a proper measurement may be obtained. As shown in FIG. 6, the bioreactor 100 may include two or more fixed beds in a stacked arrangement. The sensor system 200 may be applied to one or more (including all) of the fixed beds, as desired, including by having individual electrodes 202, 204 (e.g., probes) associated with each layer (including possibly in the form of inner and outer rings circumferentially extending along the inner and outer sides of the bed when in an annular form, as shown), or electrodes associated with a plurality of the fixed beds. Alternatively, the bioreactor may comprise a single fixed bed, as previously described, and employ one such sensor system 200.

According to a second aspect of the disclosure, measuring the biomass or cell density may be achieved by measuring a change in a parameter corresponding to the liquid flowing through the fixed bed 122 at a first point in time (such as prior to commencement of cell culturing), and then later in the process at one or more points in time. Due to the increase in cell growth over time, an indication of the cell density may be provided by the change in the measured parameter.

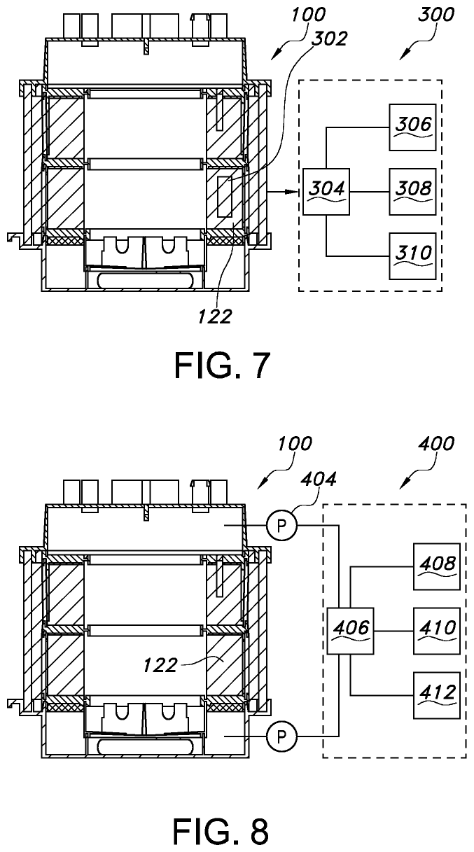

As an example, and with reference to FIG. 7, sensor system 300 is included and provides one or more liquid flowmeters 302 inside of the bioreactor 100, such as within a portion of the fixed bed 122. Such flowmeter(s) 302 permit the measurement of liquid media flow through the fixed bed 122. By measuring the liquid flow prior to the commencement of cell growth, and comparing it with flow values measured later during the cell culturing process, an indication of the cell density within the bed 122 may be obtained. For example, a decrease of the linear speed of the liquid media through the fixed bed 122 may be an indication of pressure loss in the measured region due to cell colonization in the fixed bed 122. These measurements may be used to track cell density.

The flowmeter 302 may take various forms, including but not limited to a rotary flowmeter, a Coriolis flowmeter, or an ultrasound measurement device, or any other device for measuring flow known to one skilled in the art. Regardless of the particular form, the flowmeter 302 may be optionally implemented as a single use or disposable technology. The flowmeter 302 may also be connected to an external controller 304 connected in a wired manner or wirelessly to receive the measurements, a microprocessor 306 for analyzing the data measured along with other data, an input device 308 such as a keyboard and an output device 310 for providing information in a user-perceptible form (such as a display). The microprocessor, input and output devices 306, 308, 310 may simply be in the form of a computer connected to the controller 304.

A third possible aspect of the disclosure includes a sensor system 400 as shown with reference to FIG. 8. In this version, a first pressure sensor 402 may be located adjacent an entrance end of the fixed bed 122, and a second pressure sensor 404 may be located adjacent to an exit end of the fixed bed 122. The pressure sensors 402, 404 may comprise manometers or any other device for measuring pressure known to one skilled in the art.

By measuring the pressure differential across the fixed bed 122 prior to commencement of cell culturing, and then periodically or continuously monitoring the pressure differential throughout the cell culturing process, an indication of the change in pressure may be obtained that is reflective of the cell density in the fixed bed 122. Specifically, a decrease of the pressure before and after the fixed bed 122 is an indication of the pressure loss due to cell colonization of the fixed-bed and could be used to track cell density. The pressure sensors 402, 404 may also be connected to an external controller 406 for receiving the measurements and providing information via an output device 410 in a user-perceptible form (such as via a display).

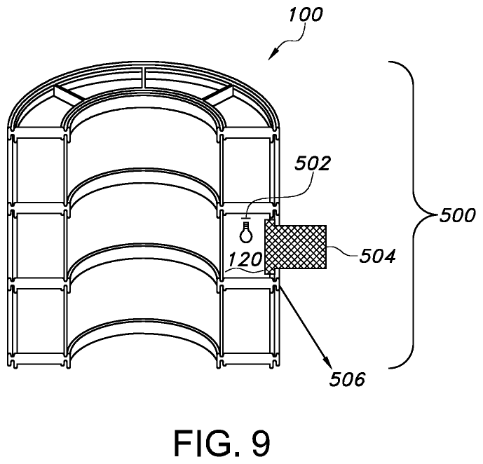

A fourth possible aspect of the disclosure includes a biomass sensor system 500 as shown with reference to FIG. 9. The material of the fixed bed 122, once colonized by cells, may have an increase in opacity. In addition, the cells have a difference in polarization and refringence as compared with the culture media. Also, the cells, due to the presence of DNA/protein, will absorb UV light at 260 and 280 nm. Hence, sensor system 500 can be based on detecting and monitoring the change of one or more of the following characteristics: refringence or polarization of light through the fixed bed 122, presence of DNA/protein (UV 260 nm and 280 nm), optical density or the turbidity, or direct observation of cells using microscopy.

In one example, one or more light sources 502 may be located inside the fixed bed 122. The light source(s) 502 may comprise an LED directly located in the bioreactor 100 adjacent to the fixed bed 122, or light may be delivered to the fixed bed from or through one or more conduits, such as optical fibers positioned within the fixed bed (removable or integral), from a light source, which may be located internal to or external to the bioreactor 100. The light source 502 may also extend along multiple fixed beds, such as in a stacked arrangement (such as for example in the case of an optical fiber or rod).

In any case, a monitor 504, such as a light sensor or microscope, may be connected to a light transmissive or transparent portion 506 of the bioreactor 100 for detecting changes in the light passing through the fixed bed 122. The sensor signals or observed output can be used to measure or extrapolate cell density. Specifically, the presence of cells in the fixed bed 122 would decrease the light transmission through the fixed bed 122 over time, and thus may provide an indication of a change in cell density.

In one alternative approach, the light source 502 may comprise a UV light source (e.g., a pulsed UV lamp). The monitor 504 may comprise a fluorescence detector, such as an attenuated total reflectance (ATR) probe insertable into the bioreactor 100. By detecting the reflectance of certain wavelengths of light, particular characteristics of the cells may be evaluated. In another approach, the light source 502 and monitor 504 may be used to measure turbidity in the culture media to provide an indication of cell density, and may be part of a single sensor located in the bioreactor 100 (e.g., a DENCYTEE sensor available from Hamilton).

While a microscope is described above, the monitor 504 may comprise a spectrometry sensor (light absorption, light extinction, fluorometry, UV light, infrared energy, Raman). The spectrometry sensor may provide macroscopic instead of (or in addition to) microscopic measurement.

Turning back to FIG. 3B, a further embodiment of a biomass sensor may comprise one or more chemical sensors 550. For example, the chemical sensor(s) 550 may comprise an electronic strip 552 located within the fixed bed 122, and associated with a controller (not shown) and output device (not shown). The strip 552 may be coated with a reactive substance, including but not limited to enzymes, mabs or chemical reagents sensitive to an aspect of the cells, such as DNA, protein, metabolites, or any similar compound(s). By measuring an output signal of the chemical sensor 550, an indication of cell density in the bed may be obtained.

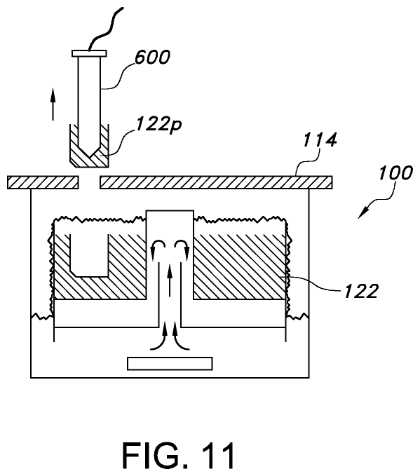

Turning to FIGS. 10 and 11, a further aspect of the disclosure pertains to a disposable single use bioreactor 100 incorporating a traditional capacitance-type biomass probe 600 in connection with a removable cell culture media. For example, in FIG. 10, the probe 600, which could be in the form of a wire, is supported by the lid or cover 114 of the bioreactor 100 formed of a plastic material and projects into the internal space of the bioreactor (e.g., through a port in the lid or cover 114), such as into the chamber including the fixed bed 122. Alternatively, such probe can pass through a port of a wall of the bioreactor.

In any case, the probe 600 is connected to a discrete representative portion 122p of the fixed bed 122, on which cells may attach and grow in like manner. The representative portion 122p may be in the form of a miniature version of the entire fixed bed 122 (such as a miniature structured fixed bed or spiral) or just a representation of a portion of such fixed bed 122 (but as should be appreciated, the larger the representative portion 122p, the more reliable the measurement in terms of being representative of the fixed bed 122). In this way, cell growth on the portion 122p is not impacted by displacement of the lid 114 due to pressure changes in the bioreactor, since the piece 122a moves together with the probe 600 during any lid or wall displacement, which may even be removed as a unit from the bioreactor 100 for inspection, as shown in FIG. 11.

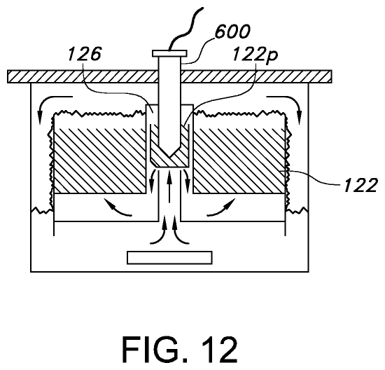

FIG. 12 illustrates an alternative embodiment where a probe 600 includes a discrete representative fixed bed material portion 122p attached at the distal end of the probe 600 corresponding to that of the fixed bed 122 in characteristics but in a smaller size. Such fixed bed material portion is positioned outside of the portion of the bioreactor 100 where the fixed bed resides so that the fixed bed is not infiltrated. In the illustrated case, the probe 600 is again supported by the lid 114, and located within a columnar central chamber 126 through which liquid cell culture media passes prior to entering the fixed bed 122. Again, this makes the discrete portion 122p of culture media attached to the probe 600 impervious to sensor signal error in the event of lid or wall displacement, since the two structures move together as a unit. This arrangement could also be used in connection with the impedance-based sensor of FIG. 4, with a portion 122p of the bed 122 connected to the electrodes 202, 204.

Furthermore, the probe 600 and portion 122p may be removed from the bioreactor 100 together, as per the FIG. 10 embodiment, to allow for closer inspection of the cell culture, if desired. One skilled in the art will understand that probe 600 can be positioned in other areas of the bioreactor where liquid media flows outside of the fixed bed region. For instance, if the flow of the bioreactor is such that the chamber outside of the fixed bed includes a liquid holding area, such probe 600 can be positioned for the portion 122p to extend therein (whether above or below the bed).

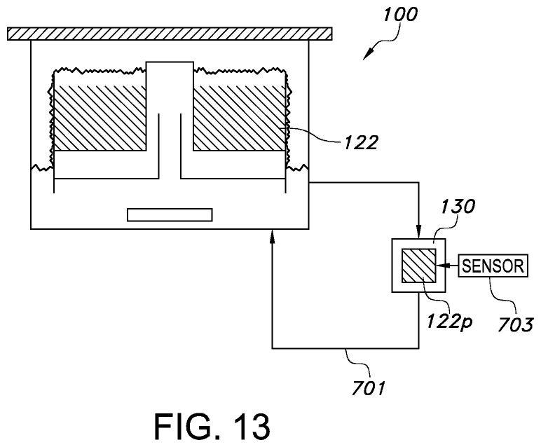

FIG. 13 illustrates an arrangement for measuring biomass using an arrangement external to the bioreactor 100. Specifically, a circulation loop 701 is provided for delivering liquid from the bioreactor 100 to a chamber 130 including a discrete representation or portion 122p of the fixed bed 122, which is within the bioreactor. In this or any of the foregoing embodiments, the discrete piece 122a may be associated with a biomass sensor 703, which may be a conventional probe arrangement, or any of the arrangements described above (for instance, impedance sensing using electrodes, flow or pressure sensing, or light sensing), but in this embodiment, the sensor is located completely external to the bioreactor 100. Again, this prevents displacements of the disposable single use bioreactor 100 from impacting the culturing of cells on the piece of culture media 122a, since it is remote from and only indirectly connected to the bioreactor.

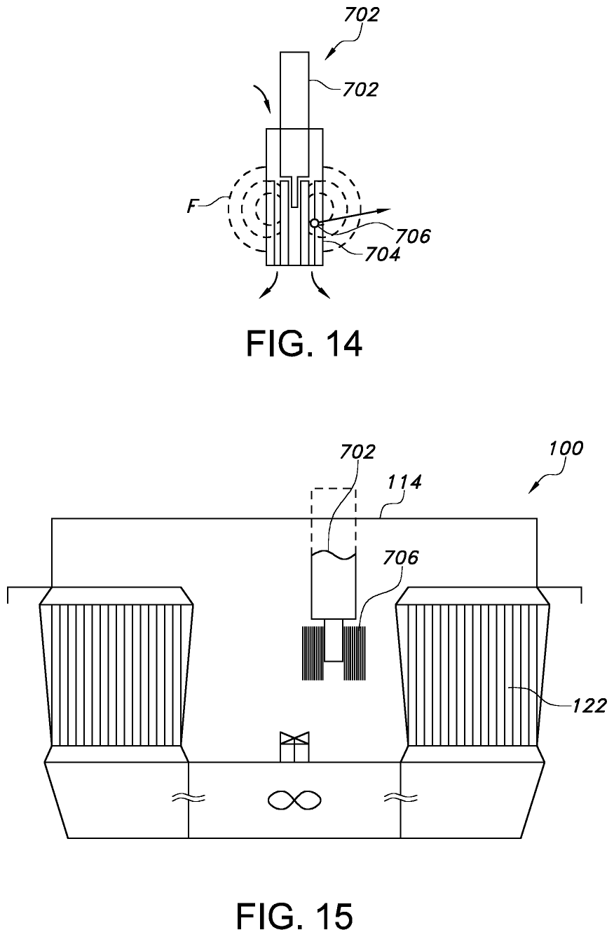

Turning to FIGS. 14 and 15, a further embodiment of a biomass sensor system 700 is disclosed. In this embodiment, a biomass sensor in the form of a traditional capacitance sensor or probe 702 supports a perforated receiver or basket 704 at one end. This basket 704 may be removably attached to the probe 702, such as by friction fit or other releasable connection. The basket 704 includes or contains a cell culture material forming an auxiliary fixed bed 706 on which cells may be cultured. This creates a “sample” fixed bed within the capacitance field F of the probe 702. Liquid cell culture media may pass through the basket 704 over the media to support cell growth in the material within the basket 704.

As shown in FIG. 15, the head end of the probe 702 may be positioned in a bioreactor 100 in the path of circulating liquid media, but in a portion separate from the fixed bed 122 (such as depending from the lid 114 into the central chamber 126, or possibly positioned within a port or well of the bioreactor) or within the fixed bed. Forming the auxiliary fixed bed 706 in the same configuration as the fixed bed 122 itself helps to ensure that similar cell growth conditions result. Thus, measuring the cell density of the auxiliary fixed bed 706 provides an indication of the cell growth conditions in the fixed bed 122, without disturbing the same. Also, because the probe 702 itself carries the auxiliary fixed bed 706, movements of the bioreactor 100 or the lid 114 do not create relative movement between the probe 702 and the material forming the fixed bed 706, and thus do not deleteriously impact measurement accuracy.

Furthermore, the releasable nature of the basket 704 allows for it to be easily removed from the probe 702 for inspection or further analysis. The independent nature of this sensor arrangement also allows for it to be assembled separately and put into use as necessary or desired, without causing the need for any variation or change in the overall design of the bioreactor 100 (since a regular sensor port could receive the probe 702) or arrangement of the fixed bed 122.

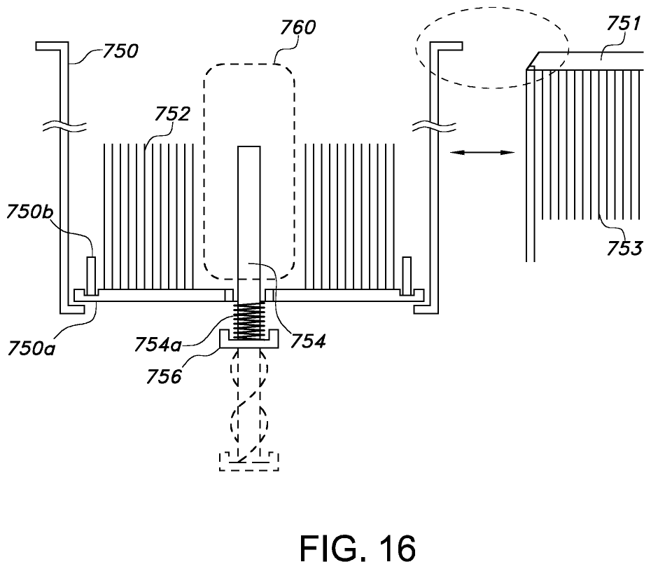

Referring now to FIG. 16, a further aspect of the disclosure is illustrated. In this aspect, a vessel 750 includes a compartment for receiving a fixed bed 752. The vessel 750 may be an auxiliary vessel in fluid communication with a bioreactor 751. The vessel 750 may also include a fixed bed 753, such as a spiral fixed bed, which may be of the same composition as the fixed bed 752. The vessel 750 may be internal or external to the bioreactor 751.

The lower portion of the vessel 750 (or a portion thereof) comprises a porous or perforated wall 750a for supporting and allowing liquid to flow through the fixed bed 752. A retainer 750b is also provided for retaining the fixed bed 752. The wall 750a and retainer 750b may be separate or unitary structures.

A piston 754 passes through the wall 750a, and includes a biasing element, such as a spring 756. In the illustrated embodiment, the spring 756 is normally biased to resist a pulling force, and thus tends to urge the piston 754 to a fully extended position within the vessel 750. A handle 754a is provided for overcoming the biasing force to partially withdraw the piston 754 from the vessel 750.

A biomass sensor or probe 760 is inserted inside the compartment by pushing vertically from top to down the piston 754. The probe 760 is applying a vertical force, overcoming the biasing force and causing the piston 754 to move vertically downward, until the probe 760 does not move anymore vertically. When required, the probe 760 can be removed, which allows for the spring 756 to recover. The operator is then able to remove the fixed bed 752, such as for an End of Process (EOP) counting cells, such as by pulling on the piston 754.

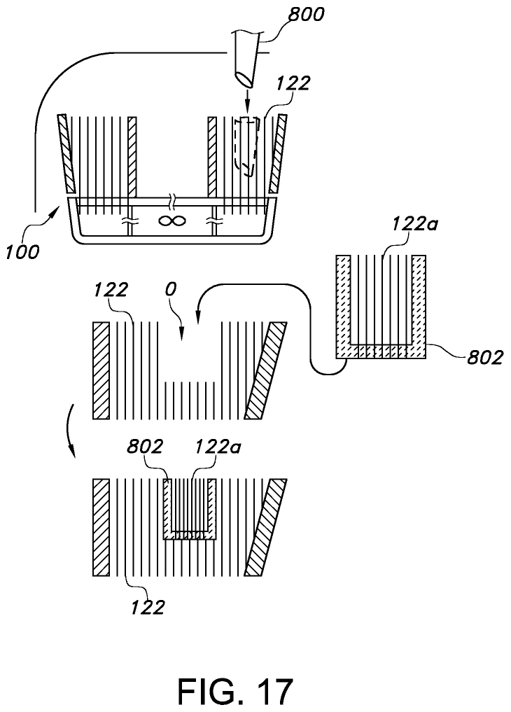

Turning to FIG. 17, this disclosure also pertains to a manner of providing a fixed bed 122 in a bioreactor 100 with a removable portion for sampling. This may involve using a cutter 800 to cut a portion 122p of the fixed bed 122, which may then be removed and placed into a receiver or basket 802, which may be perforated or porous to allow for liquid passage. The cut portion 122p and basket 802 may then together be returned to the opening O formed in the fixed bed as a result of the cutting procedure. During or after the cell culturing process, the basket 802 may be withdrawn from the fixed bed 122 to measure the cell density, without disrupting the same.

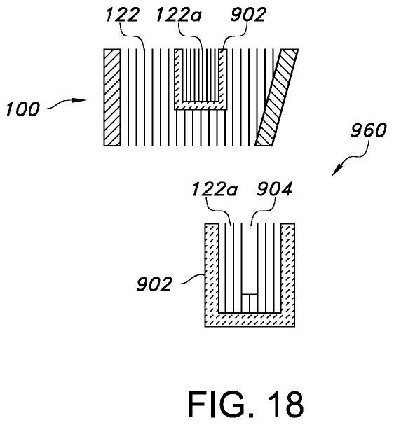

FIG. 18 further illustrates a biomass sensor arrangement 900 that has certain aspects in common with the FIG. 17 arrangement, in that a receiver or basket 902 includes a portion 122p of the fixed bed 122 in the bioreactor 100, and can be inserted into the opening created by the removal of the portion. In this case, the fixed bed portion 122p is further modified to include a passage for receiving a biomass sensor or probe 904, which then can be used to measure the biomass or cell density in the portion 122p.

In any of the disclosed embodiments, the chosen biomass sensor may be in communication with an external device, such as a controller, for receiving and displaying data indicative of the measurements taken. The arrangement may rely on direct wired communication, or may be wireless (in which case the sensor may be fully disposed in the fixed bed and thus further protected from movement of associated support structures).

Summarizing, this disclosure may relate to the following items:

1. An apparatus for culturing cells, comprising:
a bioreactor including a fixed bed for culturing cells;
a sensor system for sensing a cell density of at least a portion of the fixed bed, the sensor system selected from the group comprising:
(a) a sensor for measuring impedance across at least a portion of the fixed bed;
(b) a flowmeter for detecting a rate of flow of liquid associated with the fixed bed;
(c) a sensor for measuring a pressure differential in a flow of liquid through the fixed bed;
(d) a monitor, such as a light sensor or microscope, for detecting light from a light source for projecting light on or in the fixed bed; or
(e) a chemical sensor for detecting a chemical indicative of the cell density in the fixed bed.

2. The apparatus of item 1, wherein the sensor system comprises the sensor for measuring impedance across at least a portion of the fixed bed.

3. The apparatus of item 1 or item 2, wherein the sensor comprises a pair of electrodes arranged with the portion of the fixed bed positioned therebetween.

4. The apparatus of item 1, wherein the sensor system comprises the flowmeter for detecting a rate of flow of liquid associated with the fixed bed.

5. The apparatus of item 1 or item 4, wherein the flowmeter is located within the fixed bed.

6. The apparatus of item 1, wherein the sensor system comprises the sensor for measuring a pressure differential in a flow of liquid through the fixed bed.

7. The apparatus of item 1 or item 6, wherein the sensor comprises a first pressure sensor adjacent an entrance to the fixed bed and a second pressure sensor adjacent to an exit of the fixed bed.

8. The apparatus of any of items 6-7, wherein the sensor system comprises the monitor, such as the light sensor or microscope, for detecting light from a light source for projecting light on or in the fixed bed.

9. The apparatus of item 8, wherein the light source comprises an optical fiber extending within the fixed bed.

10. The apparatus of item 1, wherein the sensor system comprises the chemical sensor within the fixed bed for detecting a chemical indicative of the cell density in the fixed bed.

11. An apparatus for culturing cells, comprising:
a bioreactor including a fixed bed for culturing cells; and
a sensor for measuring impedance across at least a portion of the fixed bed.

12. The apparatus of item 11, wherein the sensor comprises a pair of electrodes having the portion of the fixed bed positioned therebetween.

13. An apparatus for culturing cells, comprising:
a bioreactor including a fixed bed for culturing cells; and
a flowmeter for detecting a flow rate of liquid through the fixed bed.

14. The apparatus of item 13, wherein the flowmeter is located within the fixed bed.

15. An apparatus for culturing cells, comprising:
a bioreactor including a fixed bed for culturing cells; and
a sensor for sensing a pressure differential in a flow of liquid through the fixed bed.

16. The apparatus of item 15, wherein the sensor comprises a first pressure sensor located adjacent to an entrance of the fixed bed and a second pressure sensor located adjacent to an exit of the fixed bed.

17. An apparatus for culturing cells, comprising:
a bioreactor including a fixed bed for culturing cells; and
a monitor for monitoring light from a light source for projecting light on or in the fixed bed.

18. The apparatus of item 17, wherein the light source comprises an LED for projecting light within the fixed bed.

19. The apparatus of item 17, wherein the light source comprises an optical fiber located within the fixed bed.

20. The apparatus of item 17, wherein the monitor comprises a microscope.

21. The apparatus of item 17, wherein the monitor comprises a light sensor.

22. The apparatus of item 17, wherein the light source comprises a UV lamp, and the monitor comprises a fluorescence detector.

23. An apparatus for culturing cells, comprising:
a bioreactor including a fixed bed for culturing cells; and
a chemical sensor within the fixed bed for detecting a chemical indicative of cell density in the fixed bed.

24. The apparatus of any of items 1-23, wherein the fixed bed comprises a cell growth matrix assembly having one or more cell immobilization layers having a surface which allows cells to adhere and grow, and one or more spacer layers containing a tortuous path producing structure adjacent to said cell immobilization layers, allowing passage of cells and medium along the surface of both the one or more cell immobilization and the one or more spacer layers but in a tortuous path wherein the cells will efficiently travel into the one or more cell immobilization layers and adhere at a depth therein.

25. The apparatus of any of items 1-24, wherein the bioreactor comprises an annular housing including a chamber for receiving the fixed bed.

26. The apparatus of any of items 1-25, wherein the fixed bed comprises a plurality of woven layers.

27. The apparatus of any of items 1-26, wherein the fixed bed comprises a plurality of woven layers in a vertical stack, and arranged such that a flow of liquid is in a transverse direction.

28. An apparatus for culturing cells, comprising:
a bioreactor including a fixed bed for culturing cells; and
a biomass sensor associated with a portion of the fixed bed.

29. The apparatus of item 28, wherein the portion is located in a common chamber with the fixed bed.

30. The apparatus of item 27 or item 28, wherein the portion is a representative portion of the fixed bed and is located in a chamber of the bioreactor different from the chamber including the fixed bed.

31. The apparatus of any of items 28-30, wherein the biomass sensor comprises a probe supported by a lid of the bioreactor.

32. The apparatus of any of items 28-31, wherein the portion of the fixed bed comprises a discrete piece located external to the bioreactor.

33. The apparatus of item 32, wherein the discrete piece is located in a chamber external to the bioreactor associated with a circulation loop for transmitting liquid from the bioreactor to the chamber and returning the liquid from the chamber to the bioreactor.

34. A biomass sensor including a receiver for receiving a cell culture material.

35. The biomass sensor according to item 34, wherein the receiver comprises a basket.

36. A bioreactor including a fixed bed in one chamber and the biomass sensor of any of items 1-35 in another chamber.

37. A bioreactor including a fixed bed including a cell culture material and having a liquid permeable receiver including a portion of the cell culture material of the fixed bed.

38. The bioreactor of item 37, wherein the liquid permeable receiver is located in an opening in the fixed bed formed by the removal of the portion of the cell culture material.

39. The bioreactor of item 37 or item 38, wherein the liquid permeable receiver is removable.

40. The bioreactor of any of items 37-39, wherein the portion of the cell culture material in the liquid permeable receiver includes an opening for receiving a biomass sensor.

41. A method for sensing biomass associated with a bioreactor including a fixed bed for culturing cells, comprising one or more of the following steps:
(a) measuring impedance across at least a portion of the fixed bed;
(b) detecting a rate of flow of liquid associated with the fixed bed;
(c) measuring a pressure differential in a flow of liquid through the fixed bed;
(d) detecting light from a light source for projecting light on or in the fixed bed; or
(e) detecting a chemical indicative of the cell density in the fixed bed.

42. The method of item 41, wherein the step comprises measuring impedance across at least the portion of the fixed bed.

43. The method of item 41, wherein the step comprises detecting the rate of flow of liquid associated with the fixed bed.

44. The method of item 41, wherein the step comprises measuring the pressure differential in the flow of liquid through the fixed bed.

45. The method of item 41, wherein the step comprises detecting light from a light source for projecting light on or in the fixed bed.

46. The method of item 41, wherein the step comprises detecting a chemical indicative of the cell density in the fixed bed.

47. A method for sensing biomass associated with a bioreactor including a fixed bed for culturing cells, comprising:
providing a biomass sensor at least partially within the bioreactor carrying a portion of the fixed bed.

48. The method of item 47, further including the step of removing the biomass sensor and the portion from the bioreactor.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

While preferred embodiments have been shown and described herein, it will be obvious to those skilled in the art that such embodiments are provided by way of example only. Numerous variations, changes, and substitutions will now occur to those skilled in the art without departing from the invention. For example, while the bioreactor is shown in a vertical orientation, it could be used in any orientation. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1. An apparatus for culturing cells, comprising: a bioreactor including a fixed bed for culturing cells; a sensor system for sensing a cell density of at least a portion of the fixed bed, the sensor system selected from the group comprising:
(a) a sensor for measuring impedance across at least a portion of the fixed bed;
(b) a flowmeter for detecting a rate of flow of liquid associated with the fixed bed;
(c) a sensor for measuring a pressure differential in a flow of liquid through the fixed bed;
(d) a monitor, such as a light sensor or microscope, for detecting light from a light source for projecting light on or in the fixed bed; or
(e) a chemical sensor for detecting a chemical indicative of the cell density in the fixed bed.

2. The apparatus of claim 1, wherein the sensor system comprises the sensor for measuring impedance across at least a portion of the fixed bed.

3. The apparatus of claim 2, wherein the sensor comprises a pair of electrodes arranged with the portion of the fixed bed positioned therebetween.

4. The apparatus of claim 1, wherein the sensor system comprises the flowmeter for detecting a rate of flow of liquid associated with the fixed bed.

5. The apparatus of claim 4, wherein the flowmeter is located within the fixed bed.

6. The apparatus of claim 1, wherein the sensor system comprises the sensor for measuring a pressure differential in a flow of liquid through the fixed bed.

7. The apparatus of claim 6, wherein the sensor comprises a first pressure sensor adjacent an entrance to the fixed bed and a second pressure sensor adjacent to an exit of the fixed bed.

8. The apparatus of claim 1, wherein the sensor system comprises the monitor, such as the light sensor or microscope, for detecting light from a light source for projecting light on or in the fixed bed.

9. The apparatus of claim 8, wherein the light source comprises an optical fiber extending within the fixed bed.

10. The apparatus of claim 1, wherein the sensor system comprises the chemical sensor within the fixed bed for detecting a chemical indicative of the cell density in the fixed bed.

11.-27. (canceled)

28. An apparatus for culturing cells, comprising: a bioreactor including a fixed bed for culturing cells; and a biomass sensor associated with a portion of the fixed bed.

29. The apparatus of claim 28, wherein the portion is located in a common chamber with the fixed bed.

30. The apparatus of claim 28, wherein the portion is a representative portion of the fixed bed and is located in a chamber of the bioreactor different from the chamber including the fixed bed.

31. The apparatus of claim 28, wherein the biomass sensor comprises a probe supported by a lid of the bioreactor.

32. The apparatus of claim 28, wherein the portion of the fixed bed comprises a discrete piece located external to the bioreactor.

33. The apparatus of claim 32, wherein the discrete piece is located in a chamber external to the bioreactor associated with a circulation loop for transmitting liquid from the bioreactor to the chamber and returning the liquid from the chamber to the bioreactor.

34. A biomass sensor including a receiver for receiving a cell culture material.

35. The biomass sensor according to claim 34, wherein the receiver comprises a basket.

36.-48. (canceled)
